(19) United States
(12) Patent Application Publication
(10) Pub. No.: US 2025/0246304 A1
(43) Pub. Date: Jul. 31, 2025

(54) Systems for Dynamic Personalized Healthcare Insight Generation and Conveyance

(71) Applicant: E-LOVU HEALTH, INC., Cupertino, CA (US)

(72) Inventors: Santosh Pandipati, Los Gatos, CA (US); Noel M. Pugh, San Francisco, CA (US); Terry Duesterhoeft, Aptos, CA (US)

(21) Appl. No.: 18/426,194

(22) Filed: Jan. 29, 2024

(51) Int. Cl. G16H 50/20; G16H 10/60; G16H 40/20

(52) U.S. Cl. CPC G16H 50/20; G16H 10/60; G16H 40/20

(57) ABSTRACT

A system includes a data acquisition engine that receives input data for multiple patients, including medical, situational, and environmental data, which is stored and curated within a data store. A patient assessment engine of the system processes the input data through rule-based algorithms to automatically generate a real-time patient assessment for a target patient, which is fed back into the data store. The system includes a comprehensive care artificial intelligence (AI) system that implements a real-time dynamic predictive AI model for processing data within the data store to automatically identify causal relationships pertinent to the real-time patient assessment for the target patient, and automatically generate a real-time dynamic healthcare recommendation for the target patient, which is fed back into the data store. A dashboard generator of the system provides output conveying both the real-time patient assessment for the target patient and the associated real-time dynamic recommendation for the target patient.

### CROSS-REFERENCE TO RELATED APPLICATIONS

This application is related to U.S. patent application Ser. No. 17/850,951, filed on Jun. 27, 2022, and to U.S. patent application Ser. No. 18/307,752, filed on Apr. 26, 2023, and to U.S. patent application Ser. No. 18/307,757, filed on Apr. 26, 2023. The disclosure of each above-mentioned patent application is incorporated herein by reference in its entirety for all purposes.

### BACKGROUND OF THE INVENTION

A constant drive exists to improve healthcare practices, services, and products for the benefit of patients, healthcare providers, healthcare marketplace partners, and essentially any other entity that interfaces with the healthcare industry. This drive for improvement in the healthcare industry benefits from the collective experiences of large populations of patients, healthcare providers, and healthcare marketplace partners. Healthcare providers are now also confronted with large amounts of remote patient health data from outside traditional medical practice that does not readily integrate into existing electronic medical record systems. Therefore, it is expected that the quality, efficiency, and economics of healthcare services and products can be improved by harnessing medical practice experiential knowledge across a large domain of patients, healthcare providers, and healthcare marketplace partners. However, due to the high demand for patient services and the limited amount of time available per patient encounter, healthcare providers may not be able to meaningfully and comprehensively process a large volume of patient/provider experiential data in a sufficiently fast manner to glean information that is pertinent to a time-sensitive patient care situation. Moreover, many medical professionals simply do not have the time or analytical expertise to process large volumes of patient/provider experiential data in a timely manner so as to significantly affect their healthcare decision-making process. Additionally, even if a medical professional did have the time, resources, and expertise to pour through large volumes of patient/provider experiential data in a timely manner, there may be some insights within the data that cannot be gleaned through human intellect alone. It is within this context that the present invention arises.

### SUMMARY OF THE INVENTION

In an example embodiment, a system is disclosed for surfacing dynamic personalized healthcare insight for a target patient. The system includes a data acquisition engine configured to receive input data that includes data streams of medical data for multiple patients, data streams of situational data for the multiple patients, and data streams of environmental characterization data relevant to the multiple patients. The multiple patients include the target patient. The received input data includes real-time medical data, real-time situational data, and real-time environmental data for the target patient. The data acquisition engine is configured to identify a type of the received input data and direct storage of the received input data based on the type of the received input data. The system also includes a data store configured to store the received input data as directed by the data acquisition engine. The system also includes a patient assessment engine configured to process the received input data for the target patient through a rule-based algorithm to automatically generate a real-time patient assessment for the target patient. The system is configured to feedback the real-time patient assessment for the target patient to the data acquisition engine for entry into the data store. The system also includes a comprehensive care artificial intelligence (AI) system configured to implement a real-time dynamic predictive AI model that processes data within the data store to automatically identify causal relationships pertinent to the real-time patient assessment for the target patient. The comprehensive care AI system is further configured to utilize the identified causal relationships to automatically generate a real-time dynamic healthcare recommendation pertinent to the real-time patient assessment for the target patient. The system is configured to feedback the identified causal relationships and the real-time dynamic recommendation that are pertinent to the real-time patient assessment for the target patient to the data acquisition engine for entry into the data store. The system also includes a dashboard generator configured to prepare and transmit an output data stream that provides for graphical display of output information on a computing device of a user of the system. The output information conveys both the real-time patient assessment for the target patient and the real-time dynamic recommendation pertinent to the real-time patient assessment for the target patient.

In an example embodiment, a method is disclosed for surfacing dynamic personalized healthcare insight for a target patient. The method includes receiving input data that includes data streams of medical data for multiple patients, data streams of situational data for the multiple patients, and data streams of environmental characterization data relevant to the multiple patients. The multiple patients include the target patient. The received input data includes real-time medical data, real-time situational data, and real-time environmental data for the target patient. The method also includes identifying a type of the received input data. The method also includes storing the received input data in a data store based on the type of the received input data. The method also includes processing the received input data for the target patient through a rule-based algorithm to automatically generate a real-time patient assessment for the target patient. The method also includes storing the real-time patient assessment for the target patient in the data store. The method also includes providing the real-time patient assessment for the target patient as an input to a comprehensive care AI system. The method also includes operating the comprehensive care AI system to implement a real-time dynamic predictive AI model that processes data within the data store to automatically identify causal relationships pertinent to the real-time patient assessment for the target patient. The method also includes operating the comprehensive care AI system to utilize the identified causal relationships to automatically generate a real-time dynamic healthcare recommendation pertinent to the real-time patient assessment for the target patient. The method also includes storing the identified causal relationships and the real-time dynamic recommendation that are pertinent to the real-time patient assessment for the target patient in the data store. The method also includes preparing an output data stream that provides for graphical display of output information on a remote computing device. The output information conveys both the real-time patient assessment for the target patient and the real-time dynamic recommendation pertinent to the real-time patient assessment for the target patient. The method also includes transmitting the output data stream to the remote computing device.

### BRIEF DESCRIPTION OF THE DRAWINGS

FIG. 1 shows a system for surfacing dynamic personalized healthcare insight for a target patient, in accordance with some embodiments.

FIG. 2A shows a first example portion of the graphical user interface output by the system of FIG. 1, in accordance with some embodiments.

FIG. 2B shows a second example portion of the graphical user interface output by the system of FIG. 1, in accordance with some embodiments.

FIG. 2C shows a third example portion of the graphical user interface output by the system of FIG. 1, in accordance with some embodiments.

FIG. 2D shows a fourth example portion of the graphical user interface output by the system of FIG. 1, in accordance with some embodiments.

FIG. 3 shows a flowchart of a method for surfacing dynamic personalized healthcare insight for a target patient, in accordance with some embodiments.

### DETAILED DESCRIPTION OF THE INVENTION

In the following description, numerous specific details are set forth in order to provide an understanding of the embodiments disclosed herein. It will be apparent, however, to one skilled in the art that the embodiments disclosed herein may be practiced without some or all of these specific details. In other instances, well known process operations have not been described in detail in order not to unnecessarily obscure the disclosed embodiments.

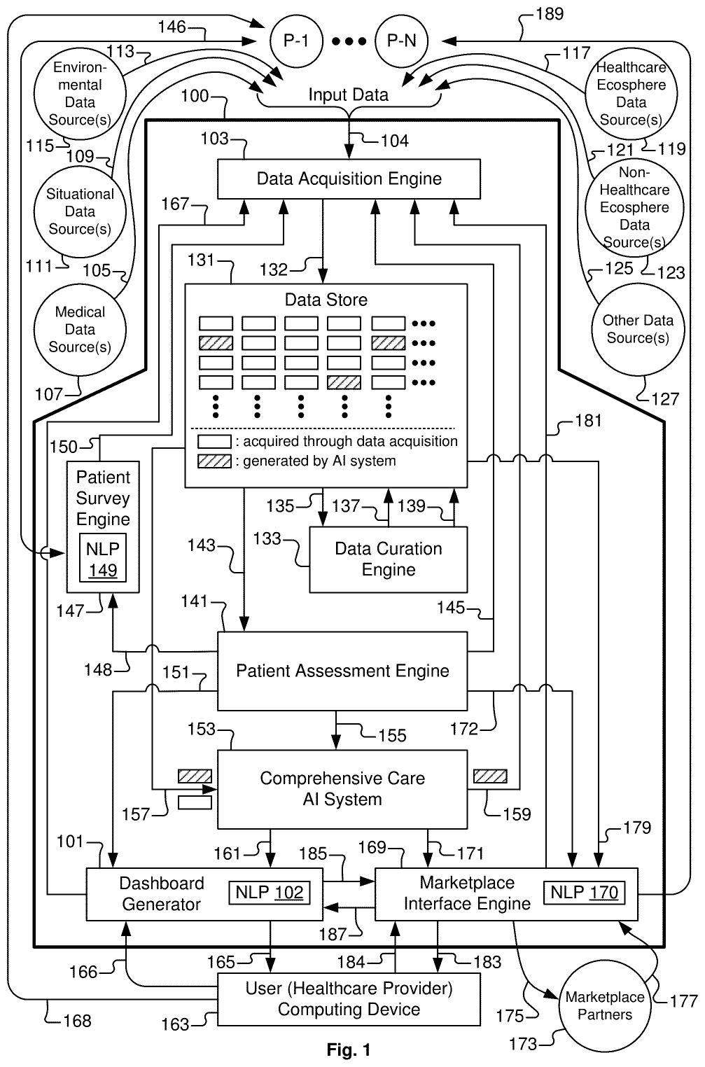

FIG. 1 shows a system 100 for surfacing dynamic personalized healthcare insight for a target patient, in accordance with some embodiments. The system 100 implements real-time, dynamic predictive modeling of relationships between data content within multiple input data streams and the health/well-being of the target patient to generate and convey insights that are relevant, dynamic, and current in order to facilitate care of the target patient. The system 100 is configured to apply a combination of artificial intelligence (AI)-based data analysis/processing and rules-based data analysis/processing to glean insight into contextual/situational conditions that affect the health/well-being of the target patient. Also, the system 100 is configured to synthesize the results of the AI-based data analysis/processing and the results of the rules-based data analysis/processing into dynamic real-time informational conveyances about the target patient's healthcare situation that are designed for human intellectual consumption by healthcare professionals who are engaged in the care of the target patient. The system 100 is further configured to expose a dynamic personalized insight dashboard 101, e.g., a graphical user interface (GUI), through which the dynamic real-time informational conveyances about the target patient's healthcare situation are provided. The GUI of the dynamic personalized insight dashboard 101 is configured in a user-navigable format that enables the user of the system 100 to quickly obtain relevant, dynamic, and current information of interest to the care of the target patient. Additionally, in some embodiments, some of the AI-based data analysis/processing results generated by the system 100 are used as feedback input data by the system 100, such that subsequent dynamic real-time informational conveyances about the target patient's healthcare situation are based in-part upon prior AI-generated information. In some embodiments, feedback to the system 100 of at least some of the AI-based data analysis/processing results generated by the system 100 is supervised to ensure that AI components of the system 100 are further trained upon relevant and beneficial data. Also, in some embodiments, feedback to the system 100 of at least some of the AI-based data analysis/processing results generated by the system 100 is done in an unsupervised manner to reduce the influence of human bias on the further training of the AI components of the system 100. Moreover, in some embodiments, feedback to the system 100 of at least some of the AI-based data analysis/processing results generated by the system 100 is done in a combination of supervised and unsupervised manners.

The system 100 includes a data acquisition engine 103 that is configured to receive input data, as indicated by arrow 104, relevant to a population of patients P-1 to P-N (where N is any non-zero integer number) from many different data sources. The population of patients P-1 to P-N includes the target patient P-T for whom the system 100 is engaged to assess and generate healthcare recommendations. In some embodiments, the data acquisition engine 103 includes a network interface card (NIC) to provide for de-packetization and extraction of input data received by the system 100. In various implementations of the system 100, the input data received by the data acquisition engine 103 can include essentially any number and type of input data streams. The data acquisition engine 103 is configured to process the received input data to extract the input data and to identify a type of the received input data. The data acquisition engine 103 is also configured to direct storage of the received input data within a data store 131 based on the type of the received input data, as indicated by arrow 132. In some embodiments, the data acquisition engine 103 implements rules-based processing for evaluating received input data and directing storage of the received input data within the data store 131. In some embodiments, the data acquisition engine 103 implements a combination of rules-based processing and AI-based processing for evaluating received input data and directing storage of the received input data within the data store 131.

In some embodiments, the input data received by the system 100 includes one or more data stream(s) 105 of medical data for the patients P-1 to P-N, including the target patient P-T, from one or more medical data source(s) 107. For example, in various embodiments, the medical data source(s) 107 include one or more remote patient monitoring device(s) for providing real-time medical data for the patients P-1 to P-N within the data stream(s) 105 of medical data. In some embodiments, the real-time medical data for a given patient P-x (where x is any of 1 to N) includes one or more of a body temperature, a heart rate, a heart rate variability, a respiration rate, a blood pressure, a fetal heart rate, a fetal movement detection, a blood oxygen saturation level, an electrocardiogram, a body weight, a body measurement, a caloric intake value, a hydration level, a glucose level, a perspiration level, a sleep score, a medical diagnosis, and a medical image, among any other type of medical data. In some embodiments, the data stream(s) 105 of medical data include images and/or videos taken of the body of the given patient P-x. In some embodiments, the system 100 is configured to receive and process the images/videos of body of the given patient P-x as input characterizing the current status of the given patient P-x. In some embodiments, the system 100 is configured to determine differences between images/videos of the body of the given patient P-x over time and correlate those differences to the other received input data as a function of time in order to identify adverse situations and generate healthcare recommendations for the given patient P-x.

In various embodiments, one or more of the medical data source(s) 107 are implemented/enabled by one or more biometric sensors worn by the given patient P-x and/or observable of the given patient P-x. Also, in various embodiments, one or more of the medical data source(s) 107 are implemented/enabled by an application executing on a personal data communication device, e.g., cell phone, of the given patient P-x, where the personal data communication device conveys medical data within the data stream(s) 105 to the system 100. Also, in various embodiments, one or more of the medical data source(s) 107 are implemented/enabled through data communication with a terrestrial-based data communication system and/or satellite-based data communication system. Also, in various embodiments, one or more of the medical data source(s) 107 are in data communication with the system 100 through a cloud network, e.g., over Internet. It should be understood that in various embodiments, the system 100 is configured to engage in data communication with essentially any type of communication system and/or network, e.g., radio, Bluetooth, cellular, WIFI, satellite, etc.

In some embodiments, the input data received by the system 100 includes one or more data stream(s) 109 of situational data for the patients P-1 to P-N, including the target patient P-T, from one or more situational data source(s) 111. For example, in various embodiments, the situational data source(s) 111 include essentially any data source that provides information about a current status of a given patient P-x. In various embodiments, the situational data source(s) 111 include one or more activity monitoring source(s) for providing current activity data for the given patient P-x, such as a global positioning system (GPS) location (e.g., latitude/longitude) of the given patient P-x (e.g., obtained from a cellphone of the given patient P-x), a route of movement/travel of the given patient P-x (GPS-based), an exercise/step tracker output for the given patient P-x, and a sleep/wake detector output for the given patient P-x, among any other type of situational data. Also, in various embodiments, the situational data source(s) 111 include one or more scheduling data source(s) for providing schedule data for the given patient P-x, such as an electronic calendar for the given patient P-x (e.g., cloud-based calendar, cell phone based calendar, etc.), among others. Also, in various embodiments, the situational data source(s) 111 include one or more communication data source(s) for providing communication data for the given patient P-x, such as a chat stream, a text message stream, an email communication, a voice communication, a video-captured sign language communication, among others. Also, in various embodiments, the situational data source(s) 111 include one or more subjective data source(s) for providing subjective data for the given patient P-x, such as a current mood of the given patient P-x, a current emotion of the given patient P-x, a current disposition of the given patient P-x, a current energy level of the given patient P-x, and a current anxiety level of the given patient P-x, among others. Also, in various embodiments, the situational data source(s) 111 include one or more financial data source(s) for providing financial data for the given patient P-x, such as account balances, spending limits, and cost sensitivity metrics, among others. Also, in various embodiments, the situational data source(s) 111 include one or more personal preference settings for the given patient P-x, such as a daily-life survey for the given patient P-x and/or a personal preferences survey of the given patient P-x. Also, in some embodiments, a data stream of real-time situational data is received for the target patient P-T from the situational data source(s) 111. In various embodiments, the real-time situations data for the target patient P-T includes one or more of a geolocation of the target patient, a listing of calendared events for the target patient, a daily schedule for the target patient, an activity currently being performed by the target patient, and any of the other above-mentioned types of situational data.

In various embodiments, one or more of the situational data source(s) 111 are implemented/enabled by one or more biometric sensors worn by the given patient P-x and/or observable of the given patient P-x. Also, in various embodiments, one or more of the situational data source(s) 111 are implemented/enabled by an application executing on a personal data communication device, e.g., cell phone, of the given patient P-x, where the personal data communication device conveys situational data within the data stream(s) 109 to the system 100.

Also, in various embodiments, one or more of the situational data source(s) 111 are implemented/enabled through data communication with a terrestrial-based data communication system and/or satellite-based data communication system. Also, in various embodiments, one or more of the situational data source(s) 111 are in data communication with the system 100 through a cloud network, e.g., over Internet.

In some embodiments, the input data received by the system 100 includes one or more data stream(s) 113 of environmental characterization data for the patients P-1 to P-N, including the target patient P-T, from one or more environmental data source(s) 115. For example, in various embodiments, the environmental data source(s) 115 include essentially any data source that provides information about a current status of the environment that may have an impact on the given patient P-x. For example, in various embodiments, the environmental data source(s) 115 include one or more weather monitoring station(s) for providing current and/or predicted weather data for the region in which the given patient P-x is currently located and/or for one or more regions through which the given patient P-x is expected/predicted to travel. In various embodiments, the weather data includes a current and/or predicted outdoor temperature, humidity, dew point temperature, barometric pressure, precipitation status, precipitation amount, heat index, wind speed, wind direction, and visibility distance, among others. In various embodiments, the environmental data source(s) 115 include one or more air quality monitoring station(s) for providing current and/or predicted air quality data for the region in which the given patient P-x is currently located and/or for one or more regions through which the given patient P-x is expected/predicted to travel. In various embodiments, the air quality data includes a current and/or predicted air quality index (AQI) value and PM_{2.5} concentration value indicating the amount of particulate matter sized at less than or equal to about 2.5 micrometers per cubic meter, among others.

Also, in various embodiments, the environmental data source(s) 115 includes one or more environmental vector data source(s) for providing data on insect and/or animal activity that impacts vector-borne disease within a region relevant to the given patient P-x, such as mosquito monitoring data, tick monitoring data, flea monitoring data, bed bug monitoring data, black-fly monitoring data, lice monitoring data, sand-fly monitoring data, triatome bug monitoring data, tsetse-fly monitoring data, aquatic snail monitoring data, and rodent monitoring data, among essentially any other vector monitoring data. Example viruses tracked by the environmental vector data source(s) include one or more of influenza, COVID-19, chikungunya, dengue, Rift Valley fever, yellow fever, Zika, Japanese encephalitis, West Nile fever, phlebotomus fever, Crimean-Congo haemorrhagic fever, and tick-borne encephalitis, among others. Example parasites tracked by the environmental vector data source(s) include one or more of lymphatic filariasis, malaria, schistosomiasis, onchocerciasis, tungiasis, leishmaniasis, Chagas disease, and African trypanosomiasis, among others. Example bacteria tracked by the environmental vector data source(s) include one or more of plague, typhus, Louse-borne relapsing fever, lyme disease, borreliosis, rickettsial diseases, and tularaemia, among others.

In various embodiments, one or more of the environmental data source(s) 115 are implemented/enabled by an application executing on a personal data communication device, e.g., cell phone, of the given patient P-x, where the personal data communication device conveys data within the data stream(s) 113 to the system 100. Also, in some embodiments, one or more sensors implemented within the personal data communication device of the given patient P-x, or connected in data communication with the personal data communication device of the given patient P-x, are used to measure and report environmental data to the system 100. In various embodiments, one or more of the environmental data source(s) 115 are implemented/enabled through data communication with the terrestrial-based data communication system and/or satellite-based data communication system. Also, in various embodiments, one or more of the environmental data source(s) 115 are in data communication with the system 100 through a cloud network, e.g., over Internet.

In some embodiments, the input data received by the system 100 includes one or more data stream(s) 117 of healthcare-related data for the patients P-1 to P-N, including the target patient P-T, from one or more healthcare ecosphere data source(s) 119. For example, in various embodiments, the healthcare ecosphere data source(s) 119 include essentially any entity within the healthcare ecosphere of the given patient P-x, including any one or more of medical healthcare provider(s), mental healthcare provider(s), therapy provider(s), general wellness provider(s), specialized wellness provider(s), medical device provider(s), pharmaceutical provider(s), health insurance provider(s), medical regulator(s), medical standards of care provider(s), patient monitoring service provider(s), by way of example. In some embodiments, the input data provided through the data stream(s) 117 to the system 100 includes the medical history of the given patient P-x and the current medical records of the patient P-x, e.g., electronic medical record (EMR), as well as specialized reporting from various medical/healthcare provider(s) who are currently or were previously engaged with the given patient P-x. Also, the current medical condition of the target patient P-T is conveyed to the system 100 through the data stream(s) 117. In various embodiments, healthcare ecosphere data source(s) 119 are in data communication with the system 100 through a cloud network, e.g., over Internet.

In some embodiments, the input data received by the system 100 includes one or more data stream(s) 121 of non-healthcare-related data for the patients P-1 to P-N from one or more non-healthcare ecosphere data source(s) 123. For example, in various embodiments, the non-healthcare ecosphere data source(s) 123 include various service providers that provide various services to the given patient P-x. In some embodiments, service providers within the non-healthcare ecosphere data source(s) 123 include traffic monitoring companies, news providers, delivery companies, cleaning companies, repair service companies, pest control companies, utility companies, phone companies, internet service companies, insurance companies, exercise centers, community centers, massage centers, spas, beauty salons, manicure/pedicure providers, tanning salons, personal shopping services, schools, churches, retreat centers, among essentially any other service provider with which the given patient P-x interfaces during their daily-life activities. Also, in various embodiments, the non-healthcare ecosphere data source(s) 123 include various retail partners that offer products to the given patient P-x, either by sale, loan, or gift. In some embodiments, retail partners within the non-healthcare ecosphere data source(s) 123 include grocery stores, department stores, restaurants, gas stations, online stores, specialty stores, among essentially any other type of retailer with which the given patient P-x interfaces during their daily-life activities. In various embodiments, the non-healthcare ecosphere data source(s) 123 are in data communication with the system 100 through a cloud network, e.g., over Internet.

In various embodiments, the system 100 is able to receive input data through essentially any number of data streams provided by essentially any number of data sources. To illustrate this flexibility, FIG. 1 shows the input data received by the system 100 as including one or more data stream(s) 125 from one or more other data source(s) 127. Examples of some other data sources 127 include an employer of the given patient P-x, one or more entertainment venues that may be frequented by the given patient P-x, one or more government offices that may be relevant to an interest of the given patient P-x (e.g., office of parks and recreation, etc.), one or more family members of the given patient P-x, an airline, a hotel, a courier, a ride service, among essentially any other data source 125 that may intersect in some way with the daily-life activity of the given patient P-x. In various embodiments, the input data received by the system 100 in regard to a given patient P-x within the population of patients P-1 to P-N includes one or more of medical records data, demographic data, personal data, medical condition data, behavioral data, remote patient monitoring (RPM) data, mental health data, healthcare provider data, environmental data, scheduling data, personal calendar data, financial data, ecosphere partner data, patient preference data, standards of care data, peer-reviewed evidence, medical studies, social determinants of health data, nutrition data, genetics/genomics data, carrier screening information, medications and supplements information, lab results, pharmacogenetics/genomics, a digital wallet of the given patient P-x, among other data. It should be appreciated that the system 100 incorporates data streams into the target patient P-T assessment process that are not part of the traditional manually performed patient assessment process.

In various embodiments, one or more portable communication devices (e.g., cell phone, laptop, tablet, smartwatch, wearable-electronics, fitness tracker, etc.) associated with a given patient P-x are used to supply one or more of the data streams 105, 109, 113, 117, 121, and 125 of input data to the system 100. In some embodiments, an application executing on a personal data communication device of the given patient P-x uses an application programming interface (API) to interface with the data acquisition engine 103 to enable provision of input data to the system 100. Also, in some embodiments, an application executing on a personal data communication device of the given patient P-x is configured to regularly convey particular types of data to the system 100 by way of a cloud network, e.g., over Internet. For example, in various embodiments, a personal data communication device of the given patient P-x executes one or more of an exercise/fitness tracking application, a physiological parameter measurement application, a biometric sensor application, a weight tracking application, a mapping application, a GPS application, a calendar application, and a messaging application, among essentially any other type of application, where each executing application uses an API for interaction with the data acquisition engine 103 of the system 100. Also, in some embodiments, the system 100 interfaces with one or more other data processing/computing systems that have information relative to the given patient P-x. For example, in some embodiments, the system 100 interfaces with one or more of a home security system, a remote monitoring camera system, a home automation system, an automobile, a remote patient monitoring device, a medical device, an in-home air monitoring device, a wearable air monitoring device, an in-home appliance, an environment control system (e.g., thermostat, humidifier, de-humidifier, air filter, etc.), among essentially any other device/system that is associated with the given patient P-x and that is capable of data communication with the data acquisition system 103 of the system 100. In various embodiments, data communication to/from the system 100 is done through a cloud network using any of a number of known network communication protocols. Also, in some embodiments, the data acquisition engine 103 is in data communication with the Internet of Things (IoT). In some embodiments, the data acquisition engine 103 is configured for data connection with one or more applications executing on a computing device of the target patient P-T, where the one or more applications provide at least some of the input data to the data acquisition engine 103.

Over time, any data stream that is part of the input data to the system 100 can be interrupted temporarily or cancelled. Also, over time, any number of new data streams can be added to the input data received by the system 100. In this manner, the system 100 is adaptable to changes in the situations, medical conditions, environmental conditions, and daily-life activities of the patients P-1 to P-N. However, for engagement of the system 100 in performing automatic patient assessment and automatic healthcare recommendation generation for the target patient P-T at a given time, it is preferable that the input data to the system 100 at the given time include at least the data stream 105 of real-time medical data for the target patient P-T, the data stream 109 of real-time situational data for the target patient P-T, and the data stream 113 of real-time environmental data for the target patient P-T.

It should be appreciated that the system 100 is scalable to adapt to changes in the lifestyle and condition of any given patient P-x in the population of patients P-1 to P-N, including the target patient P-T. For example, if the given patient P-x were to become immobile, one or more of the data streams 105, 109, 113, 117, 121, and 125 of input data may become irrelevant to the lifestyle of the given patient P-x. Or, in another example, if the given patient P-x were to go on an extended vacation to another country, one or more of the data streams 105, 109, 113, 117, 121, and 125 may become temporarily inactive, while one or more new data streams of input data may come online in data communication with the data acquisition engine 103 of the system 100. The data acquisition engine 103 is configured to automatically adapt to the data streams that are available in the input data at a given time. For example, if the personal data communication device of the given patient P-x is off at a given time so that the system 100 is not aware of the exact location of the given patient P-x at the given time, the system 100 will adapt to operate based on the most applicable data available prior to the given time, such as the most recently obtained forecast environmental data for the region in which the given patient P-x is expected to be at the given time. This type of adaptability of the system 100 applies to changes in any of the data streams 105, 109, 113, 117, 121, and 125 over time.

In some embodiments, the data acquisition engine 103 implements a data filtering system that functions to filter data within the multiple input data streams 105, 109, 113, 117, 121, and 125 to identify specific data relevant to the target patient P-T. In some embodiments, the data filtering system implements artificial intelligence, e.g., machine learning, to identify data specifically tagged/marked for the target patient P-T and/or to make predictions about data that may be relevant to the target patient P-T. In some embodiments, the data acquisition engine 103 implements artificial intelligence, e.g., machine learning, to analyze big data that is collected from the population of patients P-1 to P-N that may have characteristics similar to those of the target patient P-T. The big data analysis provides for identification of predictive patterns within the data that may be applicable to the target patient P-T.

The data store 131 is configured to store the received input data as directed by the data acquisition engine 103. The data store 131 is a digital data repository that stores and safeguards data within the system 100. In various embodiments, the data store 131 is implemented to use one or more of network-connected data storage, cloud-based data storage, distributed data storage, local data storage, physical hard-drive storage, solid-state digital data storage, virtual storage, random access memory storage, high-bandwidth memory storage, and any other type of digital data storage mechanism. Also, in various embodiments, the data store 131 is implemented to include one or more of structured data, e.g., information tables, relational databases, etc., unstructured data, e.g., emails, images, videos, audio recordings, etc., and semi-structured data, e.g., data that has added tags, keywords, and/or metadata. It should be understood that in various embodiments the data store 131 is configured to store any type of digital data. Also, in various embodiments, the data store 131 is configured to store digital data in one or more formats, including file storage format, block storage format, object storage format, among others, by way of example. In some embodiments, data is organized within the data store 131 in a manner that supports multi-criteria decision analysis of the data. It should be understood that the data store 131 is configured to include both data that is acquired from sources external to the system 100, e.g., through the input data streams, and data that is generated by the system 100, e.g., patient assessments, AI-generated data, AI-generated recommendations, marketplace interface data, etc.

In some embodiments, the system 100 includes a data curation engine 133 configured to curate the data within the data store 131. In some embodiments, the data curation engine 133 is configured to access and read data within the data store 131, as indicated by arrow 135. Also, in some embodiments, the data curation engine 133 is configured to write data to the data store 131, as indicated by arrow 137. Also, in some embodiments, the data curation engine 133 is configured to generate and transmit commands to the data store 131, as indicated by arrow 139, where the commands direct operations of the data store 131 with regard to storage of data, movement of data, archiving of data, and deletion of data, among essentially any other data management operation. In some embodiments, the data curation engine 133 is configured to implement a data curation policy on data within the data store 131. In some embodiments, the data curation policy includes rules for one or more of storing data, filtering data, parsing data, merging data, purging data, deleting data, moving data, sorting data, categorizing data, labeling data, correlating data, locking data, and unlocking data, and any other type of data-related operation. In some embodiments, the data curation engine 133 is configured to implement the data curation policy in a continuous manner, such that the data curation engine 133 is continuously assessing a compliance of the data store 131 (data within the data store 131) with a currently enforced data curation policy, and directing corrective actions as needed. In some embodiments, the data curation engine 133 is configured to implement the data curation policy in a periodic manner or a scheduled manner. In some embodiments, the system 100 exposes a control interface for the data curation engine 133 through which a user of the system 100 is able to provide and/or specify the data curation policy to be implemented by the data curation engine 133, and specify the manner in which the data curation policy is to be implemented, e.g., continuous, periodic, or scheduled. Also, in some embodiments, the data curation engine 133 is configured to extract structured data from unstructured and/or semi-structured data within the data store 131, and in turn store the extracted structured data within the data store 131. In various embodiments, the data curation engine 133 is configured to ensure that one or more specified segments/portions of data within the data store 131 are sufficiently structured for use as input by each of a patient assessment engine 141, a comprehensive care artificial intelligence (AI) system 153, and a marketplace interface engine 169 of the system 100.

The system also includes a patient assessment engine 141 that is connected to access and process data within the data store 131, as indicated by arrow 143. The patient assessment engine 141 is configured to process the received input data for the target patient P-T through one or more rule-based algorithm(s) to automatically generate a real-time patient assessment for the target patient P-T. The one or more rule-based algorithm(s) implement extant healthcare standards, guidelines, and protocols, which are made known to the system 100 by way of input data that is received, processed, and stored by the data acquisition engine 103 within the data store 131. The system 100 is configured to feedback the real-time patient assessment for the target patient P-T to the data acquisition engine 103 for entry into the data store 131, as indicated by arrow 145. For the target patient P-T presenting a particular set of conditions at a given time, the system 100 engages the patient assessment engine 141 to process the data within the data store 131 for the target patient P-T through a set of rules to develop an assessment of the target patient P-T at the given time. The patient assessment engine 141 is configured to provide the generated patent assessment for the target patient P-T to the dashboard generator 101, as indicated by arrow 151.

In some embodiments, the patient assessment engine 141 is configured to determine symptoms/conditions of the target patient P-T from the data within the data store 131. In some embodiments, the patient assessment engine 141 is configured to determine that additional information is needed from the target patient P-T in order to complete and/or improve the patient assessment. In these embodiments, the patient assessment engine 141 is configured to generate and transmit an information request directive to a patient survey engine 147 of the system, as indicated by arrow 148. The patient survey engine 147 is configured to engage in bi-directional communication with the target patient P-T, as indicated by arrow 146, to obtain additional information from the target patient P-T that is needed by the patient assessment engine 141 to complete the assessment of the target patient P-T. In various embodiments, the additional information is obtained by operating the patient survey engine 147 to ask the target patient P-T for answers to specific questions and/or to request that the target patient P-T provide specific additional information to the system 100. In some embodiments, the patient survey engine 147 implements and/or utilizes a natural language processor (NLP) 149 to facilitate the bi-directional communication with the target patient P-T. In some embodiments, the NLP is implemented by one or more AI models/systems. The additional information that is obtained from the target patient P-T by the patient survey engine 147 is provided as input to the data acquisition engine 103, as indicated by arrow 150. The data acquisition engine 103 in turn processes the additional data into the data store 131 where it is curated by the data curation engine 133 and is ultimately made available to the patient assessment engine 141, as indicated by arrow 143.

The system 100 also includes a comprehensive care artificial intelligence (AI) system 153 configured to implement a real-time dynamic predictive AI model that processes data within the data store 131, as indicated by arrow 157, to automatically identify causal relationships pertinent to the real-time patient assessment for the target patient P-T as received from the patient assessment engine 141, as indicated by arrow 155. The comprehensive care AI system 153 is further configured to utilize the identified causal relationships to automatically generate a real-time dynamic healthcare recommendation pertinent to the real-time patient assessment for the target patient P-T. The system 100 is configured to feedback the identified causal relationships and the real-time dynamic healthcare recommendation that are pertinent to the real-time patient assessment for the target patient P-T to the data acquisition engine 103, as indicated by arrow 159, for entry into the data store 131. In this manner, the data within the data store 131 includes data previously generated by the comprehensive care AI system 153. Therefore, the data processed by the real-time dynamic predictive AI model of the comprehensive care AI system 153 includes at least some data previously generated by the comprehensive care AI system 153. In this manner, in some embodiments, the system 100 is configured to implement a recursive AI paradigm in which previous AI-generated results/data feed into new and different AI-generated results/data. The comprehensive care AI system 153 is configured to provide the identified causal relationship(s) and the generated real-time dynamic healthcare recommendation(s) that are pertinent to the real-time patient assessment for the target patient P-T to the dashboard generator 101, as indicated by arrow 161.

The comprehensive care AI system 153 builds, trains, and tasks one or more AI models for the target patient P-T to learn characteristics and identify patterns, trends and insights from the data within the data store 131 that is relevant to the target patient P-T. There are various types of machine learning algorithms that can be utilized to form and improve the AI models for the target patient P-T. In various embodiments, the comprehensive care AI system 153 utilizes methods associated with supervised machine learning, unsupervised machine learning, and/or reinforced machine learning, as known in the art of artificial intelligence.

In some embodiments, the real-time dynamic predictive artificial intelligence model of the comprehensive care AI system 153 is trained on healthcare data for a given patient P-x within the population of patients P-1 to P-N, where the healthcare data for the given patient P-x includes at least the data stream 105 of medical data. In some embodiments, the training healthcare data for the given patient P-x includes: A) a record of real-time patient assessments generated by the patient assessment engine 141 for the given patient P-x as a function of time, B) a record of real-time dynamic recommendations generated by the comprehensive care AI system 153 for the given patient P-x as a function of time, C) a record of healthcare-related actions taken with regard to the given patient P-x as a function of time, and D) a record of healthcare-related outcomes with regard to the given patient P-x as a function of time.

In some embodiments, one or both of the patient assessment engine 141 and the comprehensive care AI system 153 is configured to automatically identify a problematic situation that will adversely impact the target patient P-T when left unmitigated. In these embodiments, the comprehensive care AI system 153 is configured to generate a real-time dynamic recommendation for mitigating the problematic situation. Also, in some embodiments, the comprehensive care AI system 153 is configured to automatically identify a beneficial action that will positively impact the target patient P-T when performed. In these embodiments, the comprehensive care AI system 153 is configured to generate a real-time dynamic recommendation for performing the beneficial action.

In various embodiments, the comprehensive care AI system 153 implements one or more AI model(s) to provide AI-based predictive analysis of cause-and-effect probabilistic correlations that are embedded (and often hidden) within the data stored within the data store 131, as curated by the data curation engine 133. The AI model(s) of the comprehensive care AI system 153 function to generate output data that is formable into healthcare recommendations for and/or information about the target patient P-T, which the system 100 in turns makes available to the healthcare provider of the target patient P-T. The AI model(s) of the comprehensive care AI system 153 are trained by a cumulative pool of input data amassed over time from the population of patients P-1 to P-N. In some embodiments, health outcomes for the patients P-1 to P-N across various sets of health, situational, and environment contextual data are used as feedback to train the AI model(s) of the comprehensive care AI system 153, so that the output data generated by the AI model(s) of the comprehensive care AI system 153 is directed toward a favorable outcome for the target patient P-T. In some embodiments, the comprehensive care AI system 153 implements a deep learning engine to automatically analyze the data within the data store 131, as curated by the data curation engine 133, in real-time to identify correlations within the data that are indicative of (or potentially indicative of) cause-and-effect relationships that may have a bearing on the health and/or well-being of the target patient P-T, and/or have a bearing on a health-related outcome associated with the target patient P-T, e.g., childbirth, chemotherapy, post-procedural recovery, post-operative recovery, etc.

The healthcare recommendations generated by the comprehensive care AI system 153 for the target patient P-T include essentially any type of actionable healthcare recommendation and/or any type of information that is consumable by the healthcare provider of the target patient P-T. For example, in various embodiments, the healthcare recommendations generated by the comprehensive care AI system 153 for the target patient P-T provide one or more of a recommended course of healthcare action for the target patient P-T, a reminder for the healthcare provider of the target patient P-T, a scheduling assist for the target patient P-T, a statement of advice for the healthcare provider of the target patient P-T, among others. In this manner, the system 100 provides for AI-guided healthcare of the target patient P-T that goes far beyond the integration of just the healthcare ecosphere service providers to also include integration of daily-life activities of the target patient P-T, environmental data associated with the target patient P-T, business data associated with the target patient P-T, and essentially any other type of data or data source that intersects with the daily-life and well-being of the target patient P-T.

In some embodiments, the comprehensive care AI system 153 is configured to determine whether or not one or more health status indicators for the target patient P-T are approaching or have exceeded respective predefined thresholds. In some embodiments, the comprehensive care AI system 153 is configured to determine healthcare risk predictions for the target patient P-T. In some embodiments, the comprehensive care AI system 153 is configured to determine probabilities of successful outcomes for generated healthcare recommendations for the target patient P-T. In some embodiments, the comprehensive care AI system 153 is configured to transform target data within the data store 131 into a format that is comparable with historical reference data within the data store 131, where the target data is data that concerns the target patient P-T. In some embodiments, the comprehensive care AI system 153 is configured to identify outlier data within the data store 131. In some embodiments, the comprehensive care AI system 153 is configured to compare the outlier data with historical reference data within the data store 131 to identify correlations with pathological conditions and outcomes that are relevant to the target patient P-T. In some embodiments, the comprehensive care AI system 153 is configured to determine within-variable insights and interaction effects among variables corresponding to data within the data store 131.

In some embodiments, the comprehensive care AI system 153 is configured to automatically identify a combination of conditions and/or parameters that are indicative of a potential adverse condition for the target patient P-T. Also, in some embodiments, the comprehensive care AI system 153 is configured to determine a probability of occurrence of the potential adverse condition for the target patient P-T. Also, in some embodiments, the comprehensive care AI system 153 is configured to automatically identify and recommend mitigating actions and corresponding probabilities of success for the target patient P-T.

In some embodiments, the comprehensive care AI system 153 is configured to implement an unsupervised AI model to glean insights that are not influenced by human bias. In some embodiments, the comprehensive care AI system 153 is configured to feed information back into the data store 131, such as inferred relationships between conditions/parameters and adverse conditions, by way of example. In some embodiments, the comprehensive care AI system 153 is configured to generate a healthcare recommendation that indicates what should be done for the target patient P-T at a given time. In some embodiments, the comprehensive care AI system 153 is configured to convey the AI-generated data and recommendations into the data store 131, where the AI-generated data and recommendations are personalized to the target patient P-T.

In some embodiments, the comprehensive care AI system 153 is configured to determine a probability of effectiveness for a real-time dynamic healthcare recommendation that is pertinent to the real-time patient assessment for the target patient P-T. Also, in some embodiments, the dashboard generator 101 is configured to include the probability of effectiveness for the real-time dynamic healthcare recommendation within the output data stream that is provided to the computing device 163 of the healthcare provider for the target patient P-T, as indicated by arrow 165. In some embodiments, the comprehensive care AI system 153 is configured to determine an urgency level for the real-time dynamic recommendation that is pertinent to the real-time patient assessment for the target patient P-T. In some embodiments, the dashboard generator 101 is configured to include the urgency level within the output data stream that is provided to the computing device 163 of the healthcare provider for the target patient P-T.

The system 100 combines real-world acquired data with AI-generated data to create new AI-generated data that is focused toward generation of real-time fully-informed patient assessments and patient care recommendations for the target patient P-T, which are made available for consumption by the healthcare provider of the target patient P-T as part of their normal healthcare practice. In some embodiments, the training data for the AI models of the comprehensive care AI system 153 is focused toward a particular healthcare provider, so that the output of the system 100 will over time reflect the preferences and decision making tendencies of the particular healthcare provider. In some embodiments, information on patient outcomes is also provided as input to the system 100. The patient outcomes are correlated by the system 100 to the patient healthcare recommendations that were generated and the actual real-world patient care actions that were implemented. This allows the system 100 to weight internal process pathway correlations within the AI models of the comprehensive care AI system 153 based on favorability of patient outcomes. In some embodiments, the system 100 generates multiple healthcare recommendations for the target patient P-T, with associated results descriptions and probabilities of occurrence, and with associated complication/side-effect descriptions and probabilities of occurrence. For example, in some embodiments, for depression flagging of the target patient 100, the system 100 generates healthcare recommendations that include one or more of a marketplace recommendation, a prescription recommendation, a therapy recommendation, and a nutrition recommendation.

In some embodiments, the comprehensive care AI system 153 is configured and trained to provide healthcare recommendations for the target patient P-T that encourage a particular behavior of the target patient P-T that will benefit their physical health and/or mental health, where the particular behavior may be a behavior already familiar to the target patient P-T or a new/different behavior that is automatically and originally generated as a healthcare recommendation for the target patient P-T. For example, in some embodiments, the comprehensive care AI system 153 processes the patient assessment received from the patient assessment engine 141 in conjunction with the current real-time data within the data store 131 to automatically detect a potentially adverse condition and/or situation for the target patient P-T and generate a healthcare recommendation for the target patient P-T that will mitigate and/or avoid the detected potentially adverse condition and/or situation.

It should be understood that the system 100 operates the comprehensive care AI system 153 to continuously synchronize real-time medical information for the target patient P-T with real-time environmental and situational information associated with the target patient P-T to generate AI-based healthcare recommendations for the target patient P-T in an automatic, dynamic, and real-time manner. The system 100 also operates the dashboard generator 101 to convey the generated healthcare recommendations for the target patient P-T to the healthcare provider of the target patient P-T.

The dashboard generator 101 is configured to prepare and transmit an output data stream, as indicated by the arrow 165, that provides for graphical display of output information on the computing device 163 of a user of the system 100, such as the healthcare provider of the target patient P-T. In some embodiments, the output information conveys both the real-time patient assessment for the target patient P-T as generated by the patient assessment engine 141, and the real-time dynamic recommendation pertinent to the real-time patient assessment for the target patient P-T as generated by the comprehensive care AI system 153. In some embodiments, the system includes a graphical user interface (dynamic personalized insight dashboard) configured for display on a screen associated with the computing device 163 of the user of the system 100. In some embodiments, the graphical user interface is provided through an application executing on the computing device 163 of the user of the system 100, such as through a web-browser application, an enterprise application, a special-purpose application, or another type of application. In some embodiments, the system 100 exposes an API to a patient-facing application on a computing device of the target patient P-T. In some embodiments, the system 100 exposes an API to a healthcare ecosphere-facing application on the computing device 163 of healthcare provider of the target patient P-T. In some embodiments, the system 100 exposes an API to a marketplace ecosphere-facing application on a computing device of a marketplace partner of the target patient P-T. The above-mentioned API's are configured to provide for interfacing and interaction with the system 100 by an application operating on a computing device external to the system 100.

In some embodiments, the graphical user interface includes respective regions for displaying one or more of the real-time medical data for the target patient P-T, the real-time situational data for the target patient P-T, the real-time environmental data for the target patient P-T, the real-time patient assessment for the target patient P-T, and the real-time dynamic recommendation that is pertinent to the real-time patient assessment for the target patient P-T. Also, in some embodiments, the graphical user interface provides for bi-directional communication between the system 100 and the user of the system 100, such as the healthcare provider of the target patient P-T.

The dashboard generator 101 is configured to compile and format the healthcare recommendations and related information for the target patient P-T, as generated by the patient assessment engine 141, the comprehensive care AI system 153, and the marketplace interface engine 169, for conveyance to the computing device 163 of the healthcare provider of the target patient P-T, as indicated by arrow 165. In some embodiments, the dashboard generator 101 is defined to prepare and transmit data within data packets over a cloud network, e.g., Internet, to the computing device 163 of the healthcare provider of the target patient P-T. In these embodiments, the data packets are prepared by the dashboard generator 101 in accordance with any known and available network communication protocol. In some embodiments, the dashboard generator 101 includes a NIC to provide for packetization of outgoing data to be transmitted from the system 100.

It should be understood that the system 100 does not itself provide/issue authorized healthcare directives, make medical diagnoses, or write medical-related prescriptions. Rather, the system 100 operates to provide informational support to the healthcare provider for the target patient P-T to improve/expand the knowledge base of the healthcare provider, particularly with regard to AI-generated insights into the healthcare of the target patient P-T that would not otherwise be available to the healthcare provider. The system 100 operates to gather input data, assess and analyze the input data, and generate information and recommendations for provision to various healthcare providers within the healthcare ecosphere of the target patient P-T and/or to various providers of goods and/or services with the marketplace ecosphere of the target patient P-T. It should be understood that the information and recommendations provided by the system 100 through the dashboard generator 101 and corresponding graphical user interface are continuously updated by the system 100 in real-time based on the real-time status of data within the data store 131 and based on continuous processing of the data within the data store 131 by both the patient assessment engine 141 and the comprehensive care AI system 153.

In various embodiments, the dashboard generator 101 is configured to convey many types of data through the graphical user interface, such as the current health status of the target patient P-T, comparative data analysis results pertinent to the healthcare of the target patient P-T, identified trends and/or insights pertinent to the healthcare of the target patient P-T, AI-based predictions concerning the healthcare of the target patient P-T, standard-based healthcare recommendations for the target patient P-T, and AI-based healthcare recommendations for the target patient P-T, among other types of data. In some embodiments, the data conveyed by the dashboard generator 101 through the graphical user interface includes an alert concerning the healthcare of the target patient P-T, a probability of success for a given healthcare recommendation for the target patient P-T, a probability of occurrence of side-effects associated with implementation of a given healthcare recommendation for the target patient P-T, a type and significance of side-effects associated with implementation of a given healthcare recommendation for the target patient P-T, an estimated cost associated with implementation of a given healthcare recommendation for the target patient P-T, an estimated time frame for effectiveness of a given healthcare recommendation for the target patient P-T, an estimated time frame for achieving a final outcome of a given healthcare recommendation for the target patient P-T, a difficulty level for the target patient P-T associated with implementation of a given healthcare recommendation, a difficulty level for the healthcare provider and/or marketplace partner associated with implementation of a given healthcare recommendation for the target patient P-T, among essentially any other type of data associated with the healthcare of the target patient P-T. In various embodiments, the dashboard generator 101 is configured to convey dynamic real-time personalized information about the healthcare of the target patient P-T in many ways, such as visually through the graphical user interface and/or as a written report conveyed through email or through another type of electronic messaging system, e.g., short message service (SMS). In some embodiments, the written report generated by the system 100 for the healthcare provider is a snapshot of the information provided within the graphical user interface as generated by the dashboard generator 101 for the target patient P-T at a given time.

In some embodiments, the dashboard generator 101 is configured to receive data from the computing device 163 of the healthcare provider for the target patient P-T, as indicated by arrow 166, and in turn provide the received data to the data acquisition engine 103, as indicated by arrow 167. In some embodiments, the graphical user interface provided by the dashboard generator 101 includes one or more data entry mechanisms through which input data is conveyed to the system 100. In some embodiments, the graphical user interface provides for bi-directional communication between the system 100 and the healthcare provider of the target patient P-T. In some embodiments, the graphical user interface provides a text messaging region. In some embodiments, a keyboard is surfaced within the graphical user interface in conjunction with text message region. In some embodiments, the computing device 163 of the healthcare provider of the target patient P-T is equipped with a microphone through which audible communication is provided to the system 100. In these embodiments, the data acquisition engine 103 of the system 100 includes functionality to parse and interpret audible communication received from the healthcare provider of the target patient P-T. In some embodiments, the computing device 163 of the healthcare provider of the target patient P-T is equipped with a camera through which the healthcare provider of the target patient P-T visually communicates with the system 100, such as by taking pictures and/or video. In these embodiments, the data acquisition engine 103 of the system 100 includes functionality to interpret the images/video communication received from the healthcare provider of the target patient P-T.

In some embodiments, the input data received through the dashboard generator 101 includes information about the implementation of a healthcare recommendation for the target patient P-T, such as information about whether or the healthcare recommendation was implemented, information about the outcome resulting from implementation of the healthcare recommendation, information about any side-effects associated with implementation of the healthcare recommendation, information about any obstacles to implementation of the healthcare recommendation, information about costs associated with implementation of the healthcare recommendation, among essentially any other type of information relevant to the healthcare of the target patient P-T. In some embodiments, the input data received through the dashboard generator 101 provides for tracking of outcomes of AI-generated healthcare recommendations generated by the system 100, which are in turn used by the system 100 to further train the comprehensive care AI system 153. In this manner, the actual ultimate real-world target patient P-T healthcare actions (e.g., assessment, recommendation, prescription, etc.) that is directed by the human healthcare provider becomes new real-world input data that is provided as input data to the data acquisition engine 103 of the system 100.

In some embodiments, the input data received through the dashboard generator 101 includes responses to a survey of the healthcare provider with regard to how applicable, useful, helpful, and/or accurate they found the healthcare recommendations, as generated by the system 100, to be for the target patient P-T. The survey responses received from a particular healthcare provider are used to further train the comprehensive care AI system 153, so as to align future system 100-generated healthcare recommendations more closely to the particular healthcare provider's preferences. In this manner, the system 100 is adaptable and customizable to the healthcare practice of each healthcare provider that uses the system 100.

In some embodiments, the dashboard generator 101 includes an NLP 102 configured to linguistically process the generated healthcare recommendations for the target patient P-T in a language that is suitable for the healthcare provider of the target patient P-T. In some embodiments, the NLP 102 itself is implemented by one or more AI models. In some embodiments, the NLP 102 enables dialogue between the system 100 (operating autonomously without human involvement) and the healthcare provider of the target patient P-T. In some embodiments, the NLP 102 is engaged to receive and process audible feedback data provided by the healthcare provider of the target patient P-T. Also, in some embodiments, the graphical user interface as generated by the dashboard generator 101 includes one or more mechanisms by which the computing device 163 of the healthcare provider is directed to generate and transmit a communication to the target patient P-T, as indicated by arrow 168. For example, in some embodiments, the dashboard generator 101 is configured to convey a given healthcare recommendation within the graphical user interface in conjunction with a user-activatable control that when activated will cause the computing device 163 to take an action toward implementation of the given healthcare recommendation, where the action includes a communication with the target patient P-T, as indicated by arrow 168.

The system 100 also includes a marketplace interface engine 169 configured to automatically identify one or more marketplace partners 173 for providing products and/or services relevant to implementation of the real-time dynamic healthcare recommendation for the target patient P-T that is generated by and received from the comprehensive care AI system 153, as indicated by arrow 171, and that is pertinent to the real-time patient assessment for the target patient P-T that is generated by and received from the patient assessment engine 141, as indicated by arrow 172. In some embodiments, the marketplace interface engine 169 is configured to automatically access and analyze data about the target patient P-T within the data store 131, as indicated by arrow 179, to identify a possible solution for connecting the target patient P-T with a recommended marketplace partner 173 to receive products and/or services that are relevant to implementation of the healthcare recommendations generated by the comprehensive care AI system 153. In some embodiments, the marketplace interface engine 169 is configured to perform multi-criteria decision analysis for assessment of target patient P-T status, timeliness of care, healthcare provider status, marketplace partner 173 status, etc., in order to determine and recommend available, accessible, insurance-covered, and effective healthcare options to the target patient P-T in timely manner.

In some embodiments, the marketplace interface engine 169 is implemented by rules-based algorithms. In some embodiments, the marketplace interface engine 169 is implemented by a combination of rules-based algorithms and AI-based processes. In some embodiments, data generated by the marketplace interface engine 169 is fed back to the data acquisition engine 103 for inclusion within the data store 131, as indicated by arrow 181. Also, in some embodiments, the marketplace interface engine 169 is configured to generate and transmit data communications to the computing device 163 of the healthcare provider of the target patient P-T, as indicated by arrow 183, such as through email or through another type of electronic messaging system, e.g., SMS. In some embodiments, the marketplace interface engine 169 is defined to prepare and transmit data within data packets over a cloud network, e.g., Internet, to the computing device 163 of the healthcare provider of the target patient P-T. In these embodiments, the data packets are prepared by the marketplace interface engine 169 in accordance with any known and available network communication protocol. In some embodiments, the marketplace interface engine 169 includes a NIC to provide for packetization of outgoing data to be transmitted from the system 100.

The marketplace interface engine 169 is configured to engage in bi-directional data communication with the marketplace partners 173, as indicated by arrows 175 and 177, to obtain information about the product and/or service offerings of the marketplace partners 173, the availability of the marketplace partners 173, and any other relevant information about the marketplace partners 173. In various embodiments, the marketplace interface engine 169 is configured to provide automatic, dynamic, real-time interfacing and interaction with any of the marketplace partners 173 (service providers and/or retail partners within the healthcare ecosphere data source(s) 119 and/or the non-healthcare ecosphere data source(s) 123) that may be related to the target patient P-T, such as by exchanging data related to particular goods and/or services that are needed by the target patient P-T, by specifying goods and/or services that are available for procurement by the target patient P-T along with the corresponding prices and times of availability, and by processing of transactions on behalf of the target patient P-T. In some embodiments, the marketplace interface engine 169 is configured to obtain current information on one or more identified marketplace partners 173, including information such as an identity of a given marketplace partner 173 and one or more of an availability status of an applicable product and/or service provided by the given marketplace partner 173, a proximity of the given marketplace partner 173 to the target patient P-T, a cost of the applicable product and/or service provided by the given marketplace partner 173, an insurance coverage response for the applicable product and/or service from the given marketplace partner 173, a schedule of availability of the given marketplace partner 173 for provision of the applicable product and/or service to the target patient P-T, a location of the given marketplace partner 173, and contact information for the given marketplace partner 173, among other information. In some embodiments, the marketplace interface engine 169 is configured to automatically process pre-authorizations, next available appointments, calendar integration, appointment booking, etc., between the target patient P-T and the marketplace partners 173.

In some embodiments, the marketplace interface engine 169 is configured to generate and transmit a communication to the target patient P-T, as indicated by arrow 189. For example, in some embodiments, the marketplace interface engine 169 is configured to convey information to the target patient P-T about a particular marketplace partner 173 and/or about an engagement/transaction with the particular marketplace partner 173. The marketplace interface engine 169 is in bi-directional data communication with the dashboard generator 101, as indicated by arrows 185 and 187. In some embodiments, the marketplace interface engine 169 is configured to provide current information on the one or more identified marketplace partners 173 to the dashboard generator 101 in real-time for inclusion in the output data stream that is conveyed to the computing device 163 of the healthcare provider of the target patient P-T.

In some embodiments, the marketplace interface engine 169 is configured to receive an instruction through the dashboard generator 101 (or through the graphical user interface generated by the dashboard generator 101) directing release of the information on one or more identified marketplace partners 173 to the target patient P-T. Also, in some embodiments, the marketplace interface engine 169 is configured to receive an instruction from the computing device 163 of the healthcare provider, as indicated by arrow 184. In response to receiving said instruction, the marketplace interface engine 169 is configured to provide the information on the one or more identified marketplace partners 173 to the target patient P-T. In this manner, the marketplace interface engine 169 is configured to automatically generate referrals to marketplace partners 173 for the target patient P-T based on the healthcare recommendations generated by the comprehensive care AI system 153. In some embodiments, the marketplace interface engine 169 is configured to receive an instruction through the dashboard generator 101 (or through the graphical user interface generated by the dashboard generator 101) to proceed with making a referral to a marketplace partner 173 and a corresponding communication to the target patient P-T. In this manner, the healthcare provider for the target patient P-T is able to exercise control over the marketplace partner 173 referrals that are made to the target patient P-T. In some embodiments, the marketplace interface engine 169 is configured to assess and convey to the target patient P-T how relevant and/or meaningful access to and/or engagement with a particular marketplace partner 173 would be for the target patient P-T at a particular time. Also, in some embodiments, the marketplace interface engine 169 is configured to facilitate navigation of the target patient P-T to a particular marketplace partner 173.

In some embodiments, the marketplace interface engine 169 includes an NLP 170 configured to automatically facilitate linguistic communication with marketplace partners 173, the healthcare provider of the target patient P-T, and the target patient P-T. In some embodiments, the NLP 170 itself is implemented by one or more AI models. In some embodiments, the NLP 170 enables dialogue between the system 100 (operating autonomously without human involvement) and one or more of a marketplace partner 173, the healthcare provider of the target patient P-T, and the target patient P-T. In some embodiments, the NLP 170 is engaged to receive and process audible feedback data provided by one or more of a marketplace partner 173, the healthcare provider of the target patient P-T, and the target patient P-T.

It should be appreciated that the system 100 implements artificial intelligence to consume the multiple streams 105, 109, 113, 117, 121, and 125 of input data and creatively and automatically generate healthcare recommendations and associated information in real-time for the healthcare provider of the target patient P-T. The system 100 serves to automate the processing and correlation of the input data in order to surface hidden trends and/or insights that in turn lead to automatic generation of healthcare recommendations for the target patient P-T that are made available in real-time to the healthcare provider of the target patient P-T. In some embodiments, operation of the system 100 in regard to the target patient P-T includes receiving as input an initial set of recommendations based on both a care plan established by the healthcare provider of the target patient P-T and digital biomarkers for the target patient P-T, such as race, socioeconomic status, known environmental exposures, place of residence, employment status, job duties, baseline genetic profile data, and baseline vital sign data, e.g., weight, heart rate, blood pressure, temperature, blood oxygen level, and/or any other type of vital sign data, among essentially any other type of biomarker. Also, in some embodiments in which the target patient P-T is a pregnant woman, the system 100 is configured to automatically and autonomously generate healthcare recommendations for both the target patient P-T and her child over the timespan of the pregnancy and postpartum, and provide the generated healthcare recommendations in real-time to the healthcare provider of the target patient P-T.

It should be understood and appreciated that the system 100 is able to access and process the input data that has bearing on the health of the target patient P-T in real-time in order to automatically determine and communicate healthcare recommendations and associated information to the healthcare provider of the target patient P-T in a manner that is not otherwise humanly practical or possible. The continuous, automatic, real-time synchronization and correlation of medical data and relevant external data by the system 100 in order to provide AI-based determination of the recommended course of action, advice, and/or engagement with the target patient P-T in real-time is a process that extends far beyond the capability of the human healthcare provider. Moreover, through use of the comprehensive care AI system 153 it is possible to glean insights for care of the target patient P-T that have not been previously observed by the clinical body to date and that would not have been obvious to the clinical body to date. The clinical body as it currently exists does not have the time or human capacity to manually process the vast amounts of diverse data across a large enough population of patients P-1 to P-N to glean the same insights as gleaned by the system 100.

It should also be understood and appreciated that in various embodiments the system 100 functions to surface non-obvious insights into relationships between environmental hazards and the health/well-being of the target patient P-T. For example, the system 100 functions to surface insights that are not obvious to the average physician, mother, and/or consumer of healthcare with regard to the detrimental effects that environmental hazards can have on pregnancy and overall health. Also, there is no evidence or body of knowledge to date that considers the effect that one healthcare provider has on another healthcare provider, i.e., interaction effect. However, through application of the system 100, such interaction effects can be identified and addressed as needed. For example, consider that it is known that psychiatry has a two standard deviation of impact on an outcome for the target patient P-T, and that each of physical therapy and nutrition has a given amount of impact on an outcome for the target patient P-T. However, what is unknown is the degree to which psychiatry, physical therapy, and nutrition interact with each other to create a net new outcome for the target patient P-T. It should be appreciated that the system 100 as disclosed herein is capable of identifying and characterizing interaction outcomes of multi-variate target patient P-T treatment functions that prior to the system 100 could not be feasibly identified and characterized. Additionally, the system 100 can be leveraged to develop and output digital biomarkers to establish baseline markers. Operation of the system 100 generates real world evidence to facilitate prediction, diagnosis, monitoring, and management of target patient P-T outcomes.

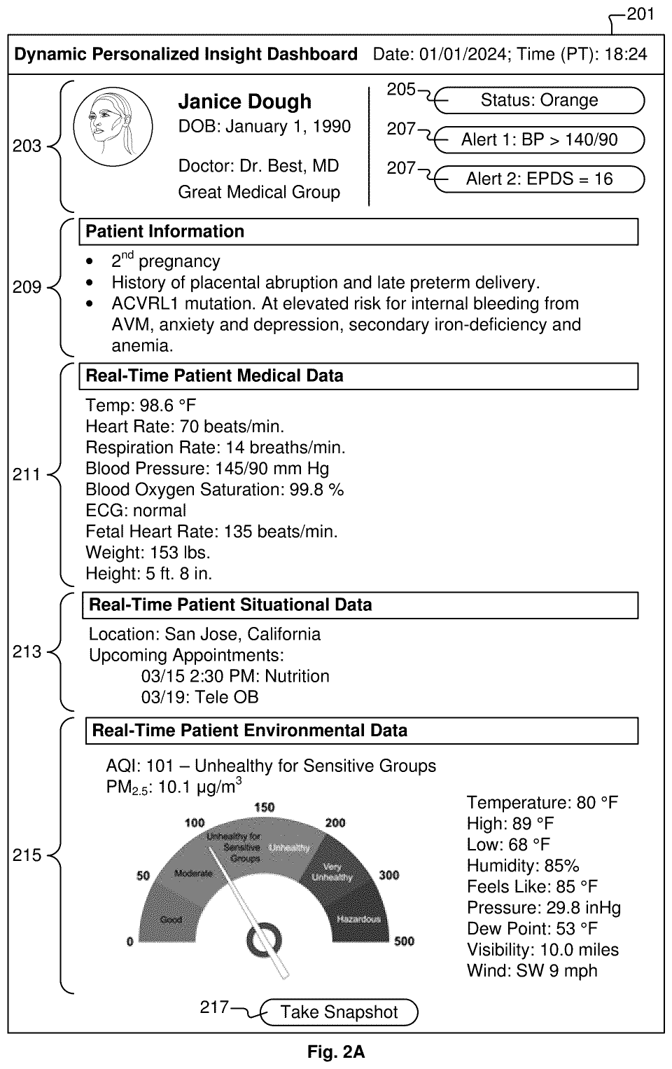

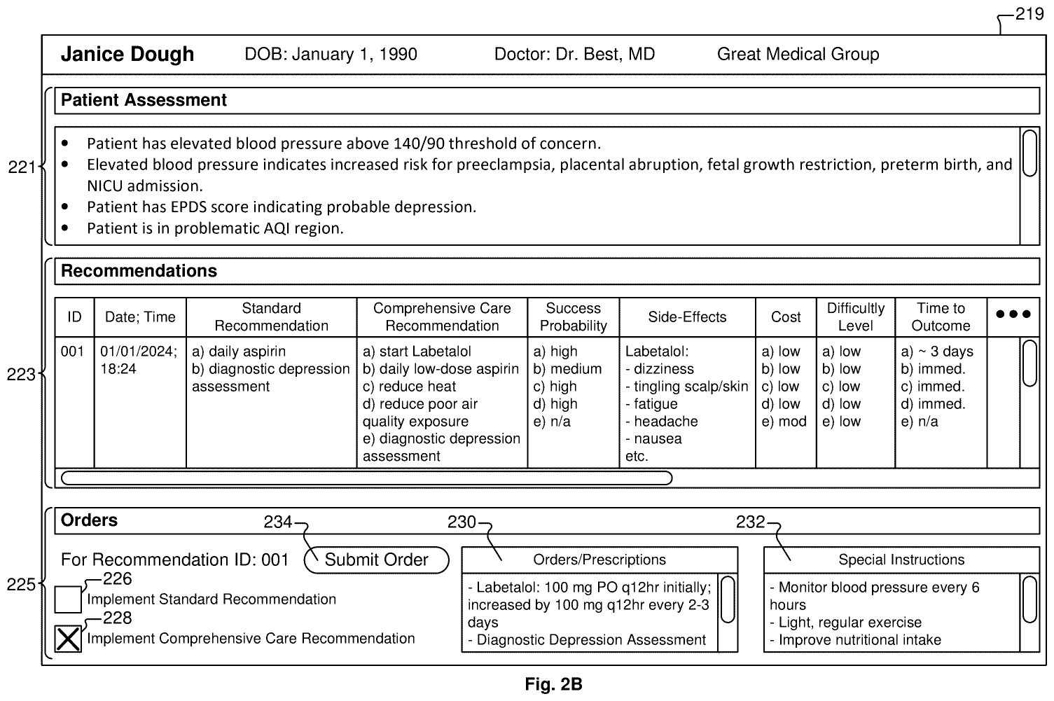

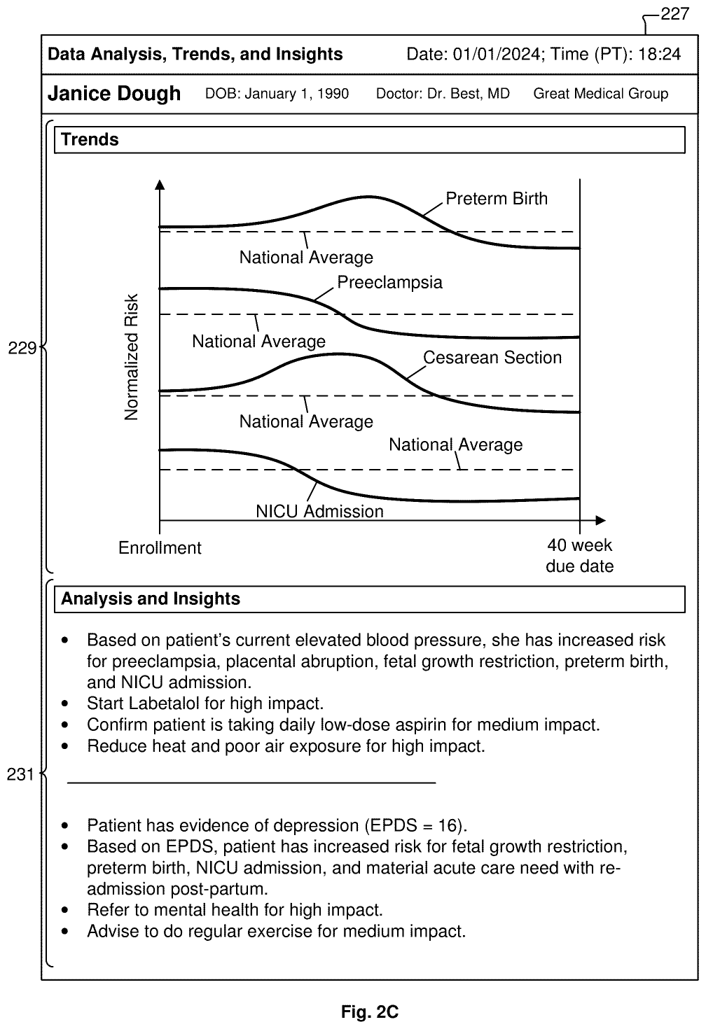

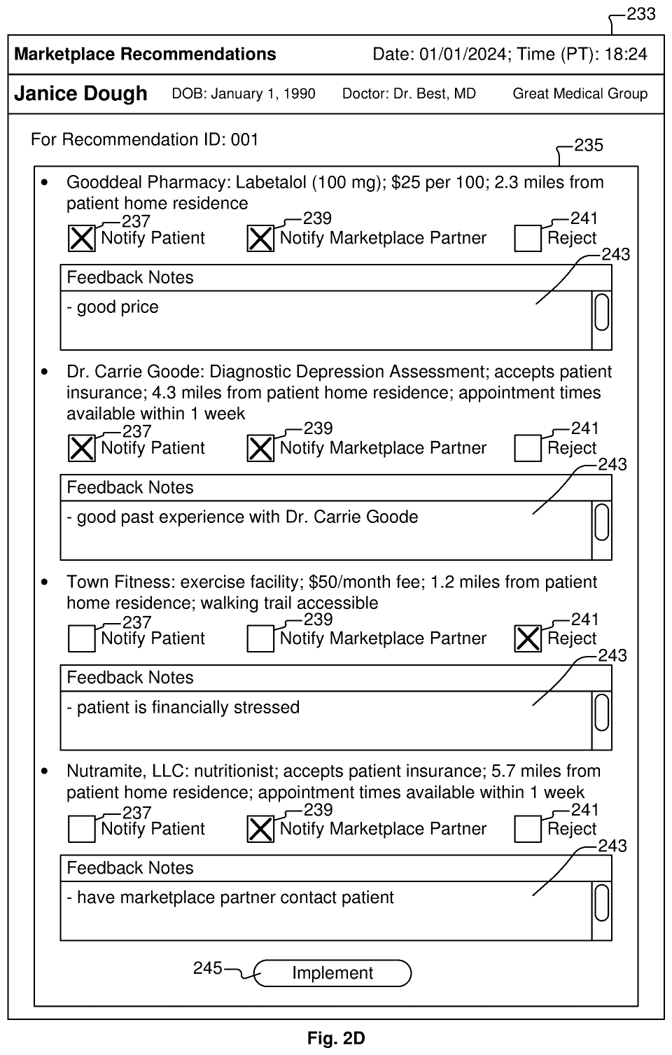

FIGS. 2A-2D show example portions of the graphical user interface provided by the dashboard generator 101 to the healthcare provider of the target patient P-T, in accordance with some embodiments. In some embodiments, the computing device 163 of the healthcare provider of the target patient P-T is equipped with a screen on which the graphical user interface is displayed. In various embodiments, the graphical user interface is defined as a dynamic personalized insight dashboard. It should be understood that the portions of the graphical user interface depicted in FIGS. 2A-2D are provided by way of example. In various embodiments, the graphical user interface provided by the dashboard generator 101 includes either less or more content than what is depicted in FIGS. 2A-2D. Also, in various embodiments, the look, feel, and user-interactivity of the graphical user interface provided by the dashboard generator 101 extends beyond and/or varies from what is shown by way of example in FIGS. 2A-2D.

FIG. 2A shows a first example portion 201 of the graphical user interface output by the system 100 of FIG. 1, in accordance with some embodiments. The first example portion 201 of the graphical user interface conveys various data regarding the current status of the target patient P-T. It should be understood that the target patient P-T data shown in FIG. 2A is provided by way of example and is in no way limiting with regard to the type of information about the status of the target patient P-T that may be conveyed through graphical user interface. In some embodiments, a patient identification region 203 is provided in which an identity of the target patient P-T is conveyed along with an identity of the healthcare provider of the target patient P-T who is authorized to access and utilize the system 100 on behalf of the target patient P-T. In various embodiments, the graphical user interface provides a login process through which the authorized healthcare provider of the target patient P-T is able to securely access the system 100. In some embodiments, the graphical user interface displays a listing the healthcare provider's patients from which a given target patient P-T is selectable so as to trigger loading and presentation of the dynamic personalized insight dashboard for the target patient P-T within the graphical user interface.

In some embodiments, a general state indicator 205 is displayed that conveys a general state of the health and/or well-being of the target patient P-T. In some embodiments, the general state indicator 205 is similar to a light that conveys a certain color that connotes a certain general state of the health and/or well-being of the target patient P-T. For example, in some embodiments, the color green in the general state indicator 205 connotes that the system 100 is determining that all is currently well with the target patient P-T. In some embodiments, the color yellow in the general state indicator 205 connotes that the system 100 is currently determining that something of relatively minor importance should be considered by the healthcare provider of the target patient P-T. In some embodiments, the color orange in the general state indicator 205 connotes that the system 100 is currently determining that something of relatively significant importance should be considered by the healthcare provider of the target patient P-T. In some embodiments, the color red in the general state indicator 205 connotes that the system 100 is currently determining that something of urgent significance should be immediately addressed by the healthcare provider of the target patient P-T. In some embodiments, the general state indicator 205 is an activatable control that, upon activation by the user (healthcare provider of the target patient P-T), will direct the graphical user interface to surface more detailed information about the basis for the currently conveyed state of the health and/or well-being of the target patient P-T.

In some embodiments, one or more alert indicators 207 are displayed to convey various types of alert information to the healthcare provider of the target patient P-T. It should be understood that the alert indicators 207 are based on current, real-time, dynamic information about the target patient P-T as gleaned by the system 100 operating on current, real-time, dynamic data within the data store 131, including AI-generated data generated by the system 100 itself. In some embodiments, each of the alert indicators 207 is an activatable control that, upon activation by the user, will direct the graphical user interface to surface more detailed information about the alert, such as the reason for the alert and any immediate actions that should be considered to resolve the alert.

In some embodiments, a patient information region 209 is shown within the graphical user interface to convey information about the current status of the target patient P-T. In various embodiments, the patient information region 209 conveys information about one or more of the current health of the target patient P-T, historical information about the target patient P-T, current diagnoses of the target patient P-T, current healthcare actions by/for the target patient P-T, potential health-related risks associated with the target patient P-T, various biomarker data about the target patient P-T, such as race, socioeconomic status, known environmental exposures, place of residence, employment status, job duties, baseline genetic profile data, among essentially any other type of information about the target patient P-T. In some embodiments, a real-time patient medical data region 209 is shown within the graphical user interface to convey information about the current medical state of the target patient P-T, such as one or more of a body temperature, a heart rate, a heart rate variability, a respiration rate, a blood pressure, a fetal heart rate, a fetal movement detection, a blood oxygen saturation level, an electrocardiogram status/result, a body weight, a body measurement, a caloric intake value, a hydration level, a glucose level, a perspiration level, a sleep score, a medical diagnosis, and a medical image, among essentially any other type of medical information about the target patient P-T.

In some embodiments, a real-time patient situational data region 213 is shown within the graphical user interface to convey information about the real-time situational data for the target patient P-T, such as one or more of a geolocation of the target patient P-T, a listing of calendared events for the target patient P-T, a daily schedule for the target patient P-T, and an activity currently being performed by the target patient P-T, among essentially any other type of situational information about the target patient P-T. In some embodiments, a real-time patient environmental data region 215 is shown with the graphical user interface to convey information about the current environmental state associated with the target patient P-T, such as one or more of an outdoor temperature value, a humidity value, a dewpoint temperature value, a barometric pressure value, an air quality index (AQI) value, a PM_{2.5} concentration value for airborne particulate matter sized at less than or equal to about 2.5 micrometers, a heat index value, a wind speed value, a wind direction, a visibility distance value, and an insect/animal vector distribution, an air quality measurement within a current vicinity of the target patient P-T, and an air quality measurement along an anticipated travel route of the target patient P-T, among essentially any other type of situational information about the target patient P-T.

In some embodiments, the graphical user interface include a snapshot control 217, that when activated/selected by the user will direct generation of a snapshot report of the current data presented within the graphical user interface. In some embodiments, the generated snapshot report is automatically emailed to the user of the system 100. In some embodiments, the generated snapshot report is written to a file that is made available for download by the user of the system 100. In some embodiments, the file containing the generated snapshot report is automatically password protected by a password uniquely associated with the user of the system 100.

FIG. 2B shows a second example portion 219 of the graphical user interface output by the system 100 of FIG. 1, in accordance with some embodiments. The second example portion 219 of the graphical user interface conveys various data regarding the assessments and recommendations that are generated by the system 100 for the target patient P-T. It should be understood that the assessment and recommendation data shown in FIG. 2B is provided by way of example and is in no way limiting with regard to the type of assessment and recommendation information for the target patient P-T that may be generated by the system 100 and conveyed through graphical user interface. In some embodiments, a patient assessment region 221 is provided in which real-time patient assessment information for the target patient P-T is conveyed. In some embodiments, the patient assessment region 221 conveys the current real-time patient assessment for the target patient P-T as currently and automatically generated by the patient assessment engine 141 and provided to the dashboard generator 101.

In some embodiments, a recommendations region 223 is provided in which real-time healthcare recommendations for the target patient P-T are conveyed. In some embodiments, the recommendations region 223 conveys the current real-time dynamic healthcare recommendation(s) that are pertinent to the real-time patient assessment for the target patient P-T as shown in the patient assessment region 221. The current real-time dynamic healthcare recommendation(s) shown in the recommendations region 223 are generated by the comprehensive care AI system 153 based on the current data within the data store 131, and are provided to the dashboard generator 101. In some embodiments, the recommendations region 223 also shows standard-based healthcare recommendation(s) that are pertinent to the real-time patient assessment for the target patient P-T as shown in the patient assessment region 221, where the standard-based healthcare recommendation(s) are generated by the system 100 based on application of one or more extant healthcare standards, guidelines, and protocols. In some embodiments, the standard-based healthcare recommendation(s) are generated by the system 100 independent of the comprehensive care AI system 153. In various embodiments, the recommendations region 223 conveys additional information about the real-time healthcare recommendations generated by the system 100 for the target patient P-T, such as one or more of a probability of success associated with implementation of a given healthcare recommendation, a probability of occurrence of side-effect(s) associated with implementation of a given health care recommendation, a description of side-effect(s) (e.g., type, significance, duration, etc.) associated with implementation of a given health care recommendation, an estimated cost of implementation of a given health care recommendation, an estimated time frame for effectiveness of a given health care recommendation, an estimated time frame for achieving a final outcome of a given health care recommendation, a difficultly level associated with implementation of a given health care recommendation (e.g., for one or more of target patient P-T, healthcare provider, and/or marketplace partner), among essentially any other type of information about the real-time healthcare recommendations generated by the system 100 for the target patient P-T.

In some embodiments, an orders region 225 is provided through which the healthcare provider is able to issue healthcare orders associated with the real-time healthcare recommendations for the target patient P-T as conveyed in the recommendations region 223. In some embodiments, the order region 225 includes a selection control 226 for directing implementation of a given standard-based healthcare recommendation as conveyed in the recommendations region 223. In some embodiments, the order region 225 includes a selection control 228 for directing implementation of a given current real-time dynamic healthcare recommendation (AI-based recommendation) as conveyed in the recommendations region 223. In some embodiments, the orders region 225 includes a input interface 230 through which the healthcare provider is able to enter orders and/or prescriptions. In some embodiments, the orders region 225 includes a input interface 232 through which the healthcare provider is able to provide special instructions for one or more of the target patient P-T, a healthcare provider, a marketplace partner, and any other entity associated with the healthcare of the target patient P-T. In some embodiments, the orders region 225 includes an order submission control 234, that when activated/selected by the user will direct the system 100 to process the corresponding order as specified in the orders region 225. In some embodiments, the system 100 is configured to engage the marketplace interface engine 169 to process the order as specified in the orders region 225.

FIG. 2C shows a third example portion 227 of the graphical user interface output by the system 100 of FIG. 1, in accordance with some embodiments. The third example portion 227 of the graphical user interface conveys various analysis, trends, and insights data generated by the system 100 with regard to the target patient P-T. It should be understood that the analysis, trends, and insights data shown in FIG. 2C is provided by way of example and is in no way limiting with regard to the types of analysis, trends, and insights information that may be generated by the system 100 and conveyed through graphical user interface. In some embodiments, a trends region 229 is provided in which one or more trends associated with the healthcare of the target patient P-T are conveyed. In some embodiments, at least some of the data shown in the trends region 229 is generated by the comprehensive care AI system 153 of the system 100. In some embodiments, an analysis and insights region 231 is provided in which various analysis and insights data associated with the healthcare of the target patient P-T is conveyed. In some embodiments, at least some of the data shown in the analysis and insights region 231 is generated by the comprehensive care AI system 153 of the system 100.

FIG. 2D shows a fourth example portion 233 of the graphical user interface output by the system 100 of FIG. 1, in accordance with some embodiments. The fourth example portion 233 of the graphical user interface conveys various marketplace recommendations generated by the system 100 with regard to the target patient P-T. It should be understood that the marketplace recommendations shown in FIG. 2D are provided by way of example and are in no way limiting with regard to the types of marketplace recommendations that may be conveyed through graphical user interface. In some embodiments, a marketplace recommendation region 235 is provided for a given healthcare recommendation as generated by the system 100 and as shown in the recommendations region 223 of FIG. 2B. In various embodiments, the marketplace recommendation region 235 includes one or more recommendations for interactions with various marketplace partners 173 that provide goods and/or services pertinent to a given healthcare recommendation for the target patient P-T. The system 100 engages the marketplace interface engine 169 to generate, organize, and provide the information within the marketplace recommendation region 235. In some embodiments, a patient notification control 237 is provided for a given marketplace recommendation (as denoted by a given bullet) that when activated/selected by the user will direct the system 100 to prepare and transmit a communication to the target patient P-T that conveys the information about the given marketplace recommendation. In some embodiments, a marketplace partner notification control 239 is provided for a given marketplace recommendation (as denoted by a given bullet) that when activated/selected by the user will direct the system 100 to prepare and transmit a communication to a marketplace partner associated with the given marketplace recommendation that conveys the information about the given marketplace recommendation in reference to the target patient P-T. In some embodiments, a reject control 241 is provided for a given marketplace recommendation (as denoted by a given bullet) that when activated/selected by the user will direct the system 100 to reject the given marketplace recommendation. In some embodiments, a feedback notes region 243 is provided for a given marketplace recommendation (as denoted by a given bullet) through which the user is able to provide feedback on the given marketplace recommendation. The system 100 is configured to store the feedback on the given marketplace recommendation within the data store 131, so that the AI models of the comprehensive care AI system 153 and/or marketplace interface engine 169 are further trained for the benefit of future-generated marketplace recommendations. In some embodiments, the marketplace recommendation region 235 for a given healthcare recommendation includes a recommendation implementation control 245, that when activated/selected by the user will direct the system 100 to implement the marketplace recommendations as specified within the marketplace recommendation region 235 for the given healthcare recommendation. In some embodiments, the system 100 is configured to engage the marketplace interface engine 169 to implement the marketplace recommendations as specified in the marketplace recommendation region 235.

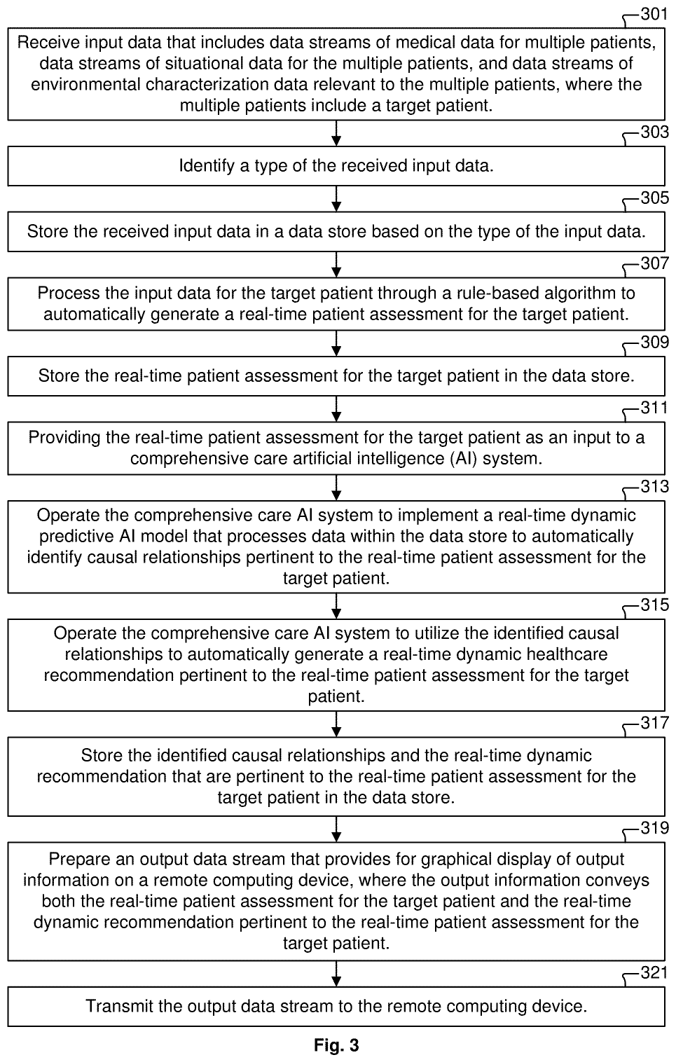

FIG. 3 shows a flowchart of a method for surfacing dynamic personalized healthcare insight for the target patient P-T, in accordance with some embodiments. In some embodiments, the method is implemented on a cloud computing system accessible over the Internet. In some embodiments, the method is implemented on one or more server computing systems accessible over the Internet. The method includes an operation 301 for receiving input data that includes data streams 105 of medical data for multiple patients P-1 to P-N, data streams 109 of situational data for the multiple patients P-1 to P-N, and data streams 113 of environmental characterization data relevant to the multiple patients P-1 to P-N. The multiple patients P-1 to P-N include the target patient P-T. The received input data includes real-time medical data, real-time situational data, and real-time environmental data for the target patient P-T. In some embodiments, the method also includes operations for receiving one or more of data streams 117 of healthcare ecosphere data for the multiple patients P-1 to P-N, data streams 121 of non-healthcare ecosphere data for the multiple patients P-1 to P-N, and data streams 125 of other data relevant to the multiple patients P-1 to P-N.

The method also includes an operation 303 for identifying a type of the received input data. The method also includes an operation 305 for storing the received input data in the data store 131 based on the type of the received input data. The method also includes an operation 307 for processing the received input data for the target patient P-T through a rule-based algorithm to automatically generate a real-time patient assessment for the target patient P-T. In some embodiments, the rule-based algorithm implements extant healthcare standards, guidelines, and protocols. The operation 307 is performed by the patient assessment engine 141. The method also includes an operation 309 for storing the real-time patient assessment for the target patient P-T in the data store 131.

The method also includes an operation 311 for providing the real-time patient assessment for the target patient P-T as an input to the comprehensive care AI system 153. The method also includes an operation 313 for operating the comprehensive care AI system 153 to implement the real-time dynamic predictive AI model that processes data within the data store 131 to automatically identify causal relationships pertinent to the real-time patient assessment for the target patient P-T. In some embodiments, the data processed by the real-time dynamic predictive AI model of the comprehensive care AI system 153 includes at least some data previously generated by the comprehensive care AI system 153. The method also includes an operation 315 for operating the comprehensive care AI system 153 to utilize the identified causal relationships to automatically generate a real-time dynamic healthcare recommendation pertinent to the real-time patient assessment for the target patient P-T. The method also includes an operation 317 for storing the identified causal relationships and the real-time dynamic recommendation that are pertinent to the real-time patient assessment for the target patient P-T in the data store 131. The method also includes an operation 319 for preparing an output data stream that provides for graphical display of output information on a remote computing device, such as the computing device 163 of the healthcare provider for the target patient P-T. In some embodiments, the output information conveys both the real-time patient assessment for the target patient P-T and the real-time dynamic recommendation pertinent to the real-time patient assessment for the target patient P-T. The method also includes an operation 321 for transmitting the output data stream to the remote computing device.

In some embodiments, the method includes training the real-time dynamic predictive AI model on healthcare data for the population of patients P-1 to P-N, where the healthcare data for a given patient P-x within the population of patients P-1 to P-N includes said data streams 105 of medical data for the given patient P-x. In some embodiments, the method includes operating the comprehensive care AI system 153 to automatically identify a problematic situation that will adversely impact the target patient P-T when left unmitigated. Also, in these embodiments, the method includes operating the comprehensive care AI system 153 to generate a real-time dynamic recommendation for mitigating the problematic situation. In some embodiments, the method includes operating the comprehensive care AI system 153 to automatically identify a beneficial action that will positively impact the target patient P-T when performed. Also, in these embodiments, the method includes operating the comprehensive care AI system 153 to generate a real-time dynamic recommendation for performing the beneficial action. In some embodiments, the method includes operating the comprehensive care AI system 153 to determine a probability of effectiveness for the real-time dynamic healthcare recommendation that is pertinent to the real-time patient assessment for the target patient P-T. Also, in these embodiments, the method includes an operation for including the probability of effectiveness for the real-time dynamic healthcare recommendation within the output information. In some embodiments, the method includes operating the comprehensive care AI system 153 to determine an urgency level for the real-time dynamic recommendation that is pertinent to the real-time patient assessment for the target patient P-T. Also, in these embodiments, the method includes an operation for including the urgency level within the output information.

In some embodiments, the method includes continuously implementing a data curation policy on data within the data store 131. The data curation policy is implemented by the data curation engine 133. The data curation policy includes rules for one or more of storing data, filtering data, parsing data, merging data, purging data, deleting data, moving data, sorting data, categorizing data, labeling data, correlating data, locking data, unlocking data, and any other type of data operation. In some embodiments, the method includes automatically identifying one or more marketplace partner(s) 173 for providing one or both of a product and a service relevant to implementation of the real-time dynamic healthcare recommendation that is pertinent to the real-time patient assessment for the target patient P-T. In some embodiments, the method includes providing current information on the one or more identified marketplace partner(s) 173 within the output information. In some embodiments, the method includes receiving an instruction directing release of the information on the one or more identified marketplace partner(s) 173 to the target patient P-T. Also, in response to receiving the instruction, the method includes providing the information on the one or more identified marketplace partner(s) 173 to the target patient P-T. The various operations concerning the marketplace partners 173 are performed at least in part by the marketplace interface engine 169.

Embodiments of the present invention may be practiced with various computer system configurations including servers, cloud systems, hand-held devices, microprocessor systems, microprocessor-based or programmable consumer electronics, minicomputers, mainframe computers and the like. The invention can also be practiced in distributed computing environments where tasks are performed by remote processing devices that are linked through a wire-based or wireless network. With the above embodiments in mind, it should be understood that the invention could employ various computer-implemented operations involving data stored in computer systems. These operations are those requiring physical manipulation of physical quantities. Usually, though not necessarily, these quantities take the form of electrical or magnetic signals capable of being stored, transferred, combined, compared and otherwise manipulated. Any of the operations described herein that form part of the invention are useful machine operations. The invention also relates to a device or an apparatus for performing these operations. The apparatus can be specially constructed for the required purpose, or the apparatus can be a general-purpose computer selectively activated or configured by a computer program stored in the computer or storage in cloud systems. In particular, various general-purpose machines can be used with computer programs written in accordance with the teachings herein, or it may be more convenient to construct a more specialized apparatus to perform the required operations. The invention can also be embodied as computer readable code on a computer readable medium. The computer readable medium is any data storage device that can store data, which can thereafter be read by a computer system. The computer readable medium can also be distributed over a network-coupled computer system so that the computer readable code is stored and executed in a distributed fashion.

The foregoing description of the embodiments has been provided for purposes of illustration and description, and is not intended to be exhaustive or limiting. Individual elements or features of a particular embodiment are generally not limited to that particular embodiment, but, where applicable, are interchangeable and can be used in a selected embodiment, even if not specifically shown or described. In this manner, one or more features from one or more embodiments disclosed herein can be combined with one or more features from one or more other embodiments disclosed herein to form another embodiment that is not explicitly disclosed herein, but rather that is implicitly disclosed herein. This other embodiment may also be varied in many ways. Such embodiment variations are not to be regarded as a departure from the disclosure herein, and all such embodiment variations and modifications are intended to be included within the scope of the disclosure provided herein.

Although some method operations may be described in a specific order herein, it should be understood that other housekeeping operations may be performed in between method operations, and/or method operations may be adjusted so that they occur at slightly different times or simultaneously or may be distributed in a system which allows the occurrence of the processing operations at various intervals associated with the processing, as long as the processing of the method operations are performed in a manner that provides for successful implementation of the method. Although the foregoing embodiments have been described in some detail for purposes of clarity of understanding, it will be apparent that certain changes and modifications can be practiced within the scope of the appended claims. Accordingly, the embodiments disclosed herein are to be considered as illustrative and not restrictive, and are therefore not to be limited to just the details given herein, but may be modified within the scope and equivalents of the appended claims.

What is claimed is:

1. A system for surfacing dynamic personalized healthcare insight for a target patient, comprising:
a data acquisition engine configured to receive input data that includes data streams of medical data for multiple patients, data streams of situational data for the multiple patients, and data streams of environmental characterization data relevant to the multiple patients, wherein the multiple patients include the target patient, the received input data including real-time medical data, real-time situational data, and real-time environmental data for the target patient, the data acquisition engine configured to identify a type of the received input data and direct storage of the received input data based on the type of the received input data;
a data store configured to store the received input data as directed by the data acquisition engine;
a patient assessment engine configured to process the received input data for the target patient through a rule-based algorithm to automatically generate a real-time patient assessment for the target patient, wherein the system is configured to feedback the real-time patient assessment for the target patient to the data acquisition engine for entry into the data store;
a comprehensive care artificial intelligence system configured to implement a real-time dynamic predictive artificial intelligence model that processes data within the data store to automatically identify causal relationships pertinent to the real-time patient assessment for the target patient, the comprehensive care artificial intelligence system further configured to utilize the identified causal relationships to automatically generate a real-time dynamic healthcare recommendation pertinent to the real-time patient assessment for the target patient, wherein the system is configured to feedback the identified causal relationships and the real-time dynamic recommendation that are pertinent to the real-time patient assessment for the target patient to the data acquisition engine for entry into the data store; and
a dashboard generator configured to prepare and transmit an output data stream that provides for graphical display of output information on a computing device of a user of the system, the output information conveying both the real-time patient assessment for the target patient and the real-time dynamic recommendation pertinent to the real-time patient assessment for the target patient.

2. The system as recited in claim 1, wherein the real-time dynamic predictive artificial intelligence model is trained on healthcare data for a population of patients, wherein the healthcare data for a given patient within the population of patients includes said data streams of medical data for the given patient.

3. The system as recited in claim 2, wherein the healthcare data for the given patient within the population of patients includes: A) a record of real-time patient assessments generated by the patient assessment engine for the given patient as a function of time, B) a record of real-time dynamic recommendations generated by the comprehensive care artificial intelligence system for the given patient as a function of time, C) a record of healthcare-related actions taken with regard to the given patient as a function of time, and D) a record of healthcare-related outcomes with regard to the given patient as a function of time.

4. The system as recited in claim 1, wherein one or both of the patient assessment engine and the comprehensive care artificial intelligence system is configured to automatically identify a problematic situation that will adversely impact the target patient when left unmitigated, and wherein the comprehensive care artificial intelligence system is configured to generate a real-time dynamic recommendation for mitigating the problematic situation.

5. The system as recited in claim 1, wherein the comprehensive care artificial intelligence system is configured to automatically identify a beneficial action that will positively impact the target patient when performed, and wherein the comprehensive care artificial intelligence system is configured to generate a real-time dynamic recommendation for performing the beneficial action.

6. The system as recited in claim 1, wherein the comprehensive care artificial intelligence system is configured to determine a probability of effectiveness for the real-time dynamic healthcare recommendation pertinent to the real-time patient assessment for the target patient, wherein the dashboard generator is configured to include the probability of effectiveness for the real-time dynamic healthcare recommendation within the output data stream.

7. The system as recited in claim 1, wherein the data acquisition engine is configured for data connection with one or more applications executing on a computing device of the target patient, wherein the one or more applications provide at least some of the input data to the data acquisition engine.

8. The system as recited in claim 1, further comprising:
a graphical user interface configured for display on the computing device of the user of the system, the graphical user interface including respective regions for displaying one or more of the real-time medical data for the target patient, the real-time situational data for the target patient, the real-time environmental data for the target patient, the real-time patient assessment for the target patient, and the real-time dynamic recommendation pertinent to the real-time patient assessment for the target patient.

9. The system as recited in claim 8, wherein the graphical user interface provides for bi-directional communication between the system and the user of the system.

10. The system as recited in claim 1, wherein the comprehensive care artificial intelligence system is configured to determine an urgency level for the real-time dynamic recommendation pertinent to the real-time patient assessment for the target patient, and wherein the dashboard generator is configured to include the urgency level within the output data stream.

11. The system as recited in claim 1, further comprising:
a data curation engine configured to continuously implement a data curation policy on data within the data store, the data curation policy including rules for one or more of storing data, filtering data, parsing data, merging data, purging data, deleting data, moving data, sorting data, categorizing data, labeling data, correlating data, locking data, and unlocking data.

12. The system as recited in claim 1, wherein the data processed by the real-time dynamic predictive artificial intelligence model of the comprehensive care artificial intelligence system includes at least some data previously generated by the comprehensive care artificial intelligence system.

13. The system as recited in claim 1, further comprising:
a marketplace interface engine configured to automatically identify one or more marketplace partner(s) for providing one or both of a product and a service relevant to implementation of the real-time dynamic healthcare recommendation pertinent to the real-time patient assessment for the target patient.

14. The system as recited in claim 13, wherein the marketplace interface engine is configured to provide current information on the one or more identified marketplace partner(s) to the dashboard generator in real-time for inclusion in the output data stream.

15. The system as recited in claim 14, wherein the current information on the one or more identified marketplace partner(s) includes an identity of a given marketplace partner and one or more of an availability status of an applicable product and/or service provided by the given marketplace partner, a proximity of the given marketplace partner to the target patient, a cost of the applicable product and/or service provided by the given marketplace partner, an insurance coverage response for the applicable product and/or service from the given marketplace partner, a schedule of availability of the given marketplace partner for provision of the applicable product and/or service to the target patient, a location of the given marketplace partner, and contact information for the given marketplace partner.

16. The system as recited in claim 14, wherein the marketplace interface engine is configured to receive an instruction directing release of the information on the one or more identified marketplace partner(s) to the target patient, and in response provide the information on the one or more identified marketplace partner(s) to the target patient.

17. The system as recited in claim 1, wherein the real-time medical data for the target patient includes one or more of a body temperature, a heart rate, a heart rate variability, a respiration rate, a blood pressure, a fetal heart rate, a fetal movement detection, a blood oxygen saturation level, an electrocardiogram, a body weight, a body measurement, a caloric intake value, a hydration level, a glucose level, a perspiration level, a sleep score, a medical diagnosis, and a medical image.

18. The system as recited in claim 1, wherein the real-time situational data for the target patient includes one or more of a geolocation of the target patient, a listing of calendared events for the target patient, a daily schedule for the target patient, and an activity currently being performed by the target patient.

19. The system as recited in claim 1, wherein the real-time environmental data for the target patient includes one or more of an outdoor temperature value, a humidity value, a dew point temperature value, a barometric pressure value, an air quality index value, a PM_{2.5} concentration value for particulate matter sized at less than or equal to about 2.5 micrometers, a heat index value, a wind speed value, a wind direction, a visibility distance value, and an insect/animal vector distribution, an air quality measurement within a current vicinity of the target patient, and an air quality measurement along an anticipated travel route of the target patient.

20. The system as recited in claim 1, wherein the target patient is one or more of a woman trying to conceive, a woman that is currently pregnant, and a woman that is within two years postpartum.
